(19) United States
(12) Patent Application Publication
(10) Pub. No.: US 2003/0134273 A1
(43) Pub. Date: Jul. 17, 2003

(54) Combined molecular binding detection through force microscopy and mass spectrometry

(76) Inventor: Eric Henderson, Ames, IA (US)

Correspondence Address: MICHAEL BEST & FRIEDRICH, LLP, ONE SOUTH PINCKNEY STREET, P O BOX 1806, MADISON, WI, 53701

(21) Appl. No.: 10/198,202

(22) Filed: Jul. 17, 2002

Related U.S. Application Data
(60) Provisional application No. 60/306,042, filed on Jul. 17, 2001.

(51) Int. Cl. C12Q 1/70; G01N 33/554; G01N 33/569; G01N 33/53; G01N 33/542

(52) U.S. Cl. 435/5; 435/7.32; 435/7.9

(57) ABSTRACT

The present invention includes a method for analyzing and characterizing molecular interaction events utilizing a combined scanning probe microscope (SPM) and a mass spectrometer (MS). An array of one or more deposition materials may be randomly deposited on a suitable surface, scanned with the SPM (or AFM) to take an initial reading of the topography of the deposition materials on the surface, and then exposed to a target sample containing a target material which may bind or interact to one or more of the deposition materials on the surface. The surface is then scanned again with the SPM to determine the molecular interaction sites and then these sites are analyzed using the MS to determine both the unknown and the deposition material.

### CROSS-REFERENCE TO RELATED APPLICATION

This application claims priority from the Provisional Patent Application, Serial No. 60/306,042 filed Jul. 17, 2001, the subject matter of which is incorporated herewith by reference for all it teaches and discloses.

### FIELD OF THE INVENTION

The present invention relates to detection and characterization of target materials utilizing a combination of scanning probe microscopy and mass spectrometry. More specifically, the present invention relates to the characterization of molecular interaction events using a combination of scanning probe microscopy and mass spectrometry.

### BACKGROUND OF THE INVENTION

A scanning probe microscope, typified by the atomic force microscope (AFM), is capable of detecting phenomena on the sub-nanometer spatial scale. The type of phenomena detected can include topography, force fields, electronic characteristics, magnetism, and a host of other interactions between the AFM probe and the sample. The type of phenomena detected is a function of the type of probe used and the methods employed.

When utilizing a standard AFM, a sharp probe scans the surface and detects the desired parameter. The measurements are so sensitive that they can be used to detect the formation of molecular complexes between as little as two molecules. For example, when an antibody binds to a protein antigen, the complex height increases. This height increase can be detected by AFM methods and the position of the complex noted.

When utilizing standard AFM methods to detect biological molecular interaction events, the scanning of the surface can be accomplished in a highly parallel array format. This is analogous to the use of fluorescence to detect nucleic acid hybridization on spotted arrays. However, a key difference in the AFM approach is that none of the interacting molecules need to be labeled with an extrinsic reporter system (i.e., no fluorescence, enzyme conjugates or radioactivity), thereby allowing use of the molecules in their native state.

Typically, when utilizing an AFM, an array is constructed that is comprised of deposition domains of a known molecular species. The array is in the form of a number of deposition domains on a surface. The AFM detects binding to any deposition domain by various molecules or molecular mixtures, indicating that an interaction has occurred between the known molecular species of the deposition domain and the diffusible molecule(s). In some embodiments, AFM detects binding by recording changes in topography of the molecular complexes. This approach has proven to be a very powerful method for analyzing large numbers of molecular interactions and correlating them with particular cell or disease states.

The current art for solid-state high throughput and highly parallel analysis of molecular interactions depends upon some prior knowledge of the identity and spatial location of the deposition domains that are on the array surface. The array may be a chip or substrate on which suitable reactive domains are placed. Each domain is comprised of one or more types (typically one) of deposition material, with as little as one domain or as many or more than a hundred domains on each array. The domains can range in size from a few nanometers to a hundred microns across. A major limitation of this approach, however, is the construction of the molecular arrays used. These arrays are usually constructed by mechanical spotting methods or by chemical synthesis directly on the array.

The above approach requires extensive manipulation and precise deposition repeatability because each domain must be in a spatially addressable format and each domain must be scannably distinct from the neighboring domains. Use of the AFM by itself can therefore limit the production and reproducibility of detecting molecular interaction events. Molecular interaction event detection becomes exponentially difficult with an increasing variety of deposition materials. Furthermore, smaller and smaller domains may be utilized in order to increase the efficiency in both time and material; however, these domains become increasingly difficult and expensive to construct.

Mass spectrometry (MS) is a method and instrument that allows a user to measure the mass of molecules and molecular fragments to characterize the same. Measuring the mass of molecules is accomplished by measuring and comparing the time it takes for an ionized molecular species to desorb from a surface, traverse a given distance and to impact a mass detector. In a standard time-of-flight MS instrument, a laser system may be employed to desorb and ionize the materials from the surface. The laser spot size determines which material is desorbed from the surface and how much of the material is desorbed. Therefore, a need exists which overcomes disadvantages of present approaches to identify materials currently in the art.

### BRIEF SUMMARY OF THE INVENTION

The present invention comprises a method for detecting, characterizing and identifying a molecular interaction event that combines the use of scanning probe microscopy and for example mass spectrometry and does not require any form of spatially organized arraying methodology. The present invention method includes making a series of randomly placed deposition domains compromised of a deposition material on a substrate to create an array. The deposition domains on the array are then scanned using the AFM probe to get a map of the surface topography or other surface characteristic of interest. The deposition domains of the array are then exposed to a target sample containing a target material. The target sample may undergo a molecular interaction event with one or more of the deposition domains. The deposition domains of the array are then scanned by the AFM probe to determine the location of the molecular interaction events, if any. The target material bound to the domain is then ionized and desorbed using highly localized desorption techniques to limit contamination by surrounding materials, possibly with the material of the deposition domain being described as well, and analyzed using a mass spectrometer to determine the identity of the target material and/or the deposition material. In the present invention method, deposition domains comprised of one or more deposition materials are randomly distributed on a surface. Because of the random distribution of the deposition domains the present invention method does not require extensive spatial addressing during deposition and array formation to characterize the unknown material.

Upon the formation and spatial location of a molecular complex, mass spectrometry allows one to measure the mass components of a desorbed complex and to deduce the identity of an unknown molecular species bound to a known target molecular species.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

The accompanying figures illustrate the components of the present invention apparatus.

The accompanying figures illustrate the components of the present invention apparatus.

FIG. 1 is a flow chart representing the present invention method.

FIG. 2 is a representative side view of a first scan of the surface during the first scan and the topography image of a deposition domain.

FIG. 3 is a representative side view of the second scan of the surface and the topography image of a molecular interaction event.

FIG. 4 is a representative side view of the desorption of the deposition material.

### DETAILED DESCRIPTION

The present invention is a method for utilizing scanning probe microscopy for molecular interaction detection, and more specifically, atomic force microscopy for molecular interaction detection, that does not require construction of numerous spatially addressable deposition domains on the array. The present invention accomplishes this by combining force microscopy with the molecular characterization by known analytical methods such as mass spectrometry.

In one embodiment the present invention utilizes the AFM to detect topographical changes in the domains. The AFM can detect and indicate to the MS the location to be desorbed and analyzed. In a further embodiment described herein the AFM can desorb the material from the surface and the MS can be solely utilized for analysis of the desorbed material.

The present method utilizes an array that is comprised of one or more deposition domains made of one or more deposition materials. The array may be referred to as a chip; both the term “array” and “chip” refer to the substrate, surface, and deposition domains placed on the same. The construction of arrays and a description of their usefulness are described in co-pending U.S. application Ser. Nos. 09/574,519 and 09/519,271 which are herein incorporated by reference for all that they teach and disclose.

The following are some definitions that may be helpful in understanding the description of the present invention. These are intended as general definitions and should in no way limit the scope of the present invention to those terms alone, but are put forth for a better understanding of the following description.

Deposition Material: This is a known material placed on a surface in a deposition domain that can be recognized and/or reacted with by a target material. The deposition material will ideally have a change inflicted upon it by one or more target materials that can be detected by later scanning with the AFM. Examples of deposition materials include, but are not limited to, biomolecules, proteins, a variety of chemicals, DNA, RNA, antibodies, or any other substance recognized by one skilled in the art which may have usefulness within the teaching of the present invention. The deposition material may be alternatively referred to as the “bait.”

Deposition Domain: A deposition domain is a spot on a surface upon which a deposition material is placed. The domain may be of any size; shape, and pattern and may contain as little as one molecule of the deposition material. These deposition domains may alternatively be referred to as “spots,” “points,” “lines,” or “patterns.” The boundary of the domain is defined by the boundary of the material placed therein.

Array: Alternatively referred to using the term “bioarray” or “molecular array,” the term array is used to describe the one or more molecular domains deposited on the surface of the substrate. The array is utilized herein to refer to the entire combination of the deposition domains, the substrate, and the surface before, after, or during the construction of the deposition domains and the exposure of the domains to the target sample.

Target Sample: A substance that contains a target material. These target samples may be natural or man-made substances. The target sample may be a solution, gas, or other medium. The target sample may likewise be artificially made or, in the alternative, a biologically produced product. Furthermore, each target sample can contain zero, one, or more target materials.

Target Material: The material detected by the present invention method. The target material can be any material with a particular affinity for one or more deposition domains. The target material may be a known or unknown entity that is present in the target sample. The target material may bind to the deposition material in the deposition domain or simply alter the deposition material in some other cognizable way. Examples of target materials may include, but are not limited to, antibodies, drugs, nucleic acids, proteins, cellular extracts, etc.

SPM: Scanning probe microscopes (SPMs) are a class of instrument that involves scanning a probe of some sort over a sample and recording interactions between the probe and sample. Usually the probe is on the microscopic spatial scale. The atomic force microscope (AFM) emphasized in this specification is one embodiment of SPM. The invention, however, is not limited for use with one specific type of SPM, but can also be incorporated for use with SPM's of various makes, models, and technological improvements. Other types of SPM may include, but are not limited to: near field scanning optical microscopy (NSOM), scanning tunneling microscopy (STM), scanning ion conductance microscopy (SICM), scanning thermal microscopy (SThM), scanning acoustic microscopy (SAM).

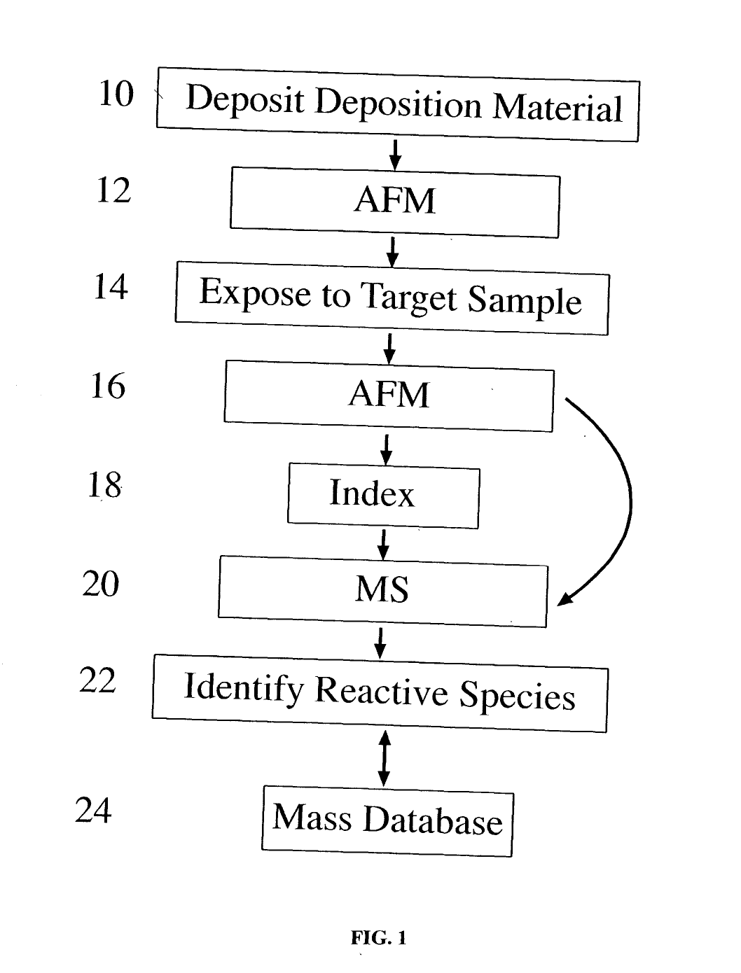

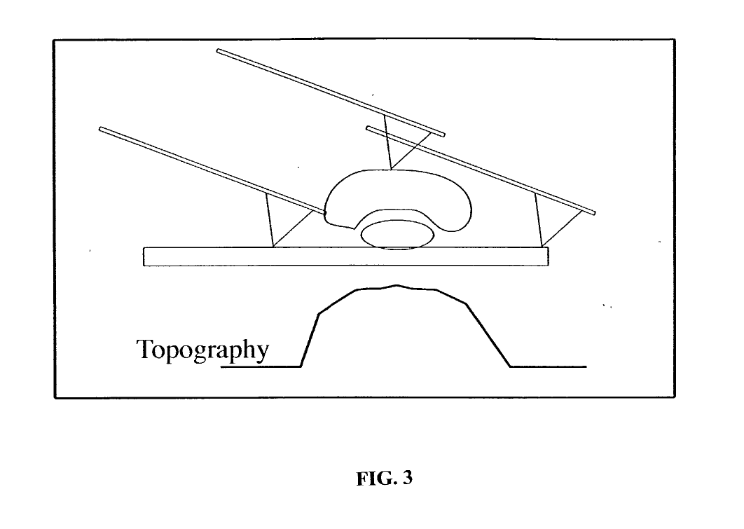

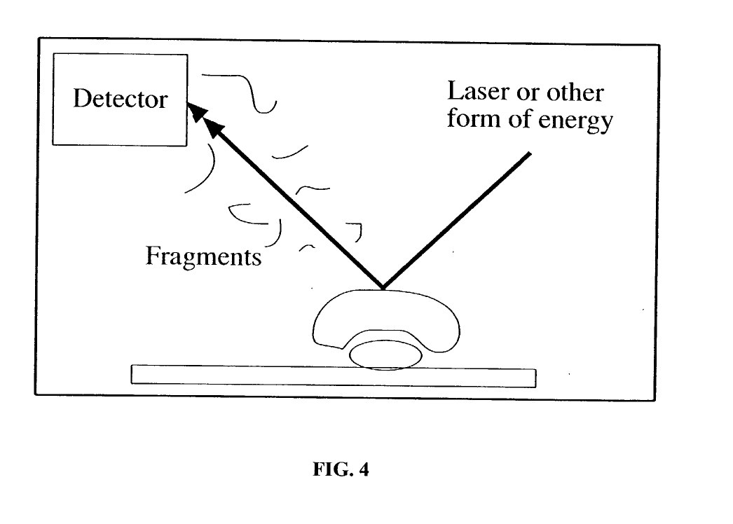

A general description of the method of the present invention will be first described herein with reference to FIGS. 1-4. After that, a few specific examples of applications of that method will be described.

Surface Preparation

A substrate for formation of an array is first provided (8), the substrate including a surface. The surface of the substrate may have another material deposited thereon to take advantage of various surface characteristics. The surface used should facilitate the deposition of the deposition material, scanning by the AFM instrument, and also be compatible with MS methods.

The substrate utilized in the present embodiment is a glass slide such as Borofloat™ glass available from United States Precision Glass, 1900-T Holmes Rd., Elgin, Ill. 60123. In another embodiment, other substrates can include, but are not limited to, mica, silicon, and quartz. Each of these materials may present various surface chemistries useful in the method.

The surface utilized is relatively smooth so that it is compatible with both the AFM and MS methodologies. Surface smoothness, however, is a relative value. For example, cleaved mica can present atomically flat surfaces over many square microns. Hansma, H. G., R. L. Sinsheimer, et al. (1992). Atomic force microscopy of single- and double-stranded DNA. Nucleic Acids Research. 20: 3585-90. Rankin, P. C. and A. T. Wilson (1969). “The Surface Chemistry of the Mica-Aluminum-Sulfate System.” 30(3): 277-282. However, mica is not an optimal substrate for the present invention because of variable quality of available mica material in addition to the fact that it can “delaminate,” resulting in loss or disruption of the surface. Loss of the surface can result in removal of the sample along with the surface and failure of the test. The Borofloat™ glass used herein is not atomically flat. Neither is highly polished silicon. However, the surface roughness of these materials is sufficiently low that the AFM is able to detect changes in height on the order of one nanometer or less. Thus, these surfaces are adequate for the detection of changes in height of a single molecule.

It is possible to use rougher surfaces and still practice the method described herein. For example, a surface with many nanometers of roughness over one to several square microns can be imaged by AFM both before and after the interrogation process. In this case, subtracting the first image from the second image results in a “subtraction map” of the surface, revealing areas in which changes in topography have occurred as a result of the molecular binding event. It is more preferable, however, to use a surface that is relatively flat compared to the height of the deposition material and the potential target sample in order to maximize the repeatability of the experiments.

The smoothness required of the underlying substrate may be a function of the sensitivity requirement of a particular test. For example, detection of a virus particle binding to antibodies on a surface requires only the smoothness of a typical glass cover slip, i.e., no “smooth” covering of the glass substrate is required. In contrast, detection of binding of a small ligand to a surface immobilized protein may require a supporting substrate with a surface roughness of one nanometer over an area of several microns. Only a carefully constructed smooth surface can achieve such smoothness.

In the present embodiment, the surface of the glass substrate may be covered with a freshly sputtered layer of gold, silver, copper, platinum, chromium, nickel, or other metals. The vacuum evaporation or ion beam sputtering methods for deposition of gold onto a surface are well known by those reasonably skilled in the art. Sputtering gold may produce a smooth surface upon which a variety of chemistries may be performed. In one embodiment, coating smooth glass (e.g., Borofloat™) with 3 nm of chromium followed by 30 nm of gold using an ion beam sputtering technique (with gold in the 10 nm grain size) produces a useful surface. A self-assembled monolayer (SAM) of a short alkane molecule (typically 11 to 18 carbons long) can then be attached to the gold layer Troughton, E., C. Bain, et al. (1988). Monolayer films prepared by the spontaneous self-assembly of symmetrical and unsymmetrical dialkyl sulfides from solution onto gold substrates: Structure, properties, and reactivity of constituent functional groups. Langmuir. 4:365-385. The alkane molecule can have a sulfhydral group at one end and a distil reactive group (e.g., succinimide, amino, carboxyl, aldehyde, epoxide, aryl azide, etc.) group at the other end. The sulfur atom binds tightly to the sputtered gold surface and the alkanes interact laterally to create a stable monolayer surface with a preferred surface chemistry, such as, for example, carboxyl, amino, succinimide, or other chemistries that facilitate attachment. Molecules of interest including, but not limited to, proteins, nucleic acids, ligands, enzymes, antibodies, sugars, lipids, can be chemically tethered to the distal reactive group by a number of chemical strategies, e.g., a condensation reaction (e.g., using EDAC for the NH2 or COOH surfaces) or using a spontaneous reaction with the succinimide group.

The gold and subsequent surface chemistries may also be created on smooth silicon, quartz or other flat surfaces.

Biomaterials can also be bound to a surface by non-specific interactions, such as by physisorption. In such an instance, a fraction of the bound biomaterial is inactive. For example, direct adsorption of antibodies to freshly sputtered gold surfaces results in bio-reactive surfaces on which the antibodies specifically bind to target material, such as an antigen, that are present in the target sample (S. Nettikadan, unpublished results).

In another embodiment, the surface may be chemically modified such that it has defined chemical properties that facilitate more defined binding interactions, such as, for example, non-covalent (e.g., ionic or hydrophobic) or covalent coupling. One method for modification of the surface in this way is to treat the glass surface with a silane containing a useful distal chemical group such as an amino or carboxyl group. These surfaces are generally somewhat rough on the nanometer scale but may be suitable for many applications utilizing the present invention. A variety of commercially available silane compounds (usually liquids) exist for this purpose. The protocols for creating these surfaces are readily available (e.g., Hulls catalog). Aminopropyltriethoxysilane (APTES) is one commonly used silane for this purpose Shlyakhtenko, L. S., A. A. Gall, et al. (1999). Atomic force microscopy imaging of DNA covalently immobilized on a functionalized mica substrate. Biophysical Journal. 77: 568-576.

Formation of the Deposition Domain

One or more deposition materials may be deposited to form the randomly placed deposition domains. First, pure samples of deposition material, typically in a physiological solution, are provided. Second, these deposition materials are made into small crystals or aggregations of the materials that are suitable for deposition on the sample substrate. Each crystal or aggregation is homogeneous with respect to a particular molecular species. This aspect of the invention allows the material to form a domain that is uncontaminated by other molecular species and would confuse the AFM and analytical characterization such as MS analysis.

In one embodiment, the deposition material is first consolidated into a crystal or otherwise amalgamated form to create micron sized particle of the deposition material, possibly containing thousands of the same molecular species. To crystallize the deposition material, the deposition material is placed in a hanging drop or a sealed tube in the presence of various solvents and other nucleating materials that facilitate the formation of biological crystals. Durbin, S. D. and G. Feher (1996). “Protein crystallization.” Annual Review of Phys Chemistry 47: 171-204.

Crystallization is necessary to obtain the three-dimensional structure of proteins and nucleic acids; it often represents the bottleneck in structure determination. Our understanding of crystallization mechanisms is still incomplete. Protein-protein contacts in crystals are complex, involving a delicate balance of specific and nonspecific interactions. Depending on solution conditions, these interactions can lead to nucleation of crystals or to amorphous aggregation; this stage of crystallization has been successfully studied by light scattering. Post-nucleation crystal growth may proceed by mechanisms involving crystal defects or two-dimensional nucleation, as observed by atomic force and interference microscopy. Cessation of growth has been observed but remains incompletely understood. Impurities may play important roles during all stages of crystallization. Phase diagrams can guide optimization of conditions for nucleation and subsequent crystal growth; a theoretical understanding relating these to the intermolecular interactions is beginning to develop.

This art form is widely practiced and there are virtually endless combinations of materials and preparations that are employed to achieve crystal growth. In other approaches, the biomaterials to be deposited are crystallized following any of a variety of known crystallization methods. The end result of these efforts is the formation of microscopic or macroscopic crystals that contain the purified biological material.

Creating the crystals or amalgams is done with each desired deposition material. A number of different crystals formed of different deposition materials can then be randomly deposited onto the substrate by utilization of an aerosol spray.

An alternative approach of putting the deposition material on the surface includes spraying the deposition material using an atomizer or similar device into a container of liquid nitrogen where the surface has been placed. When the atomized micro-droplets contact the liquid nitrogen they instantly freeze and descend to the bottom of the container. A random distribution of the atomized micro-droplets results on the surface. By carrying out this process with a variety of materials that are to be used as deposition materials, it is possible to create a densely packed, randomly distributed surface containing all the deposited materials in deposition domains. Maintenance of the array with the deposition materials thereon in liquid nitrogen may retain the biological activity of the deposition materials indefinitely since freezing in liquid nitrogen is a preferred method for long term storage of biomaterials. Furthermore, while submerged in liquid nitrogen the chip is impervious to contamination from the surrounding atmosphere.

Once the deposition materials are randomly deposited on the surface, the deposition materials (the crystallized microparticles of the deposition material) are dissolved in place with an appropriate solvent to form the deposition domains. The dissolution of the microparticles of deposition material forms mono or multi-layers at a particular position on the surface. In other words, when the deposited deposition material microparticles are dissolved in place, domains of the dissolved material are formed. Since the deposition material microparticle was initially homogeneous, the deposition domain thus formed will be essentially homogeneous. The periphery of the deposition domain, where the deposition domain may interact with an adjacent deposition domain of a second deposition material, may be locally non-homogenous along the perimeter, i.e., the deposition domains may have some degree of local mixing along a perimeter that touches a deposition domain of a different deposition material. In this way a mosaic of the various biomaterials is created on the surface.

To dissolve the deposition materials to form the deposition domains, the deposition materials may be hydrated in situ. The hydration step is carried out by raising the local humidity by placing the array (i.e., the substrate with the deposition domain(s) thereon) in a humidity controlled environment. In the embodiment wherein the deposition material is placed on the surface of the substrate using liquid nitrogen, the liquid nitrogen is allowed to evaporate before the array is placed in a humidity controlled environment. Alternatively, the substrate can be removed from the liquid nitrogen manually and placed in said humidity controlled environment.

Humidity control can be accomplished using a commercial “humidifier” or some other device for controlling the local humidity. For example, a glass tube containing an inlet and outlet may be fitted with an absorbent material such as a sponge or filter paper that has been saturated with water. Air or other gas (e.g., argon) may be passed through the chamber to create a humid stream that may be used to humidify the sample. The level of humidity may be controlled by using a valving mechanism to regulate the flow of wet and dry gas onto the sample. Alternative methods of humidity control are also useful.

The end result of this process is a surface containing homogeneous pools or domains of a variety of biomaterials. The size of these domains depends upon a number of variables including: particle size, humidity level, duration of humidification, and affinity of the biomaterial for the surface. The exposure time among the components can be from seconds to hours, sufficient to permit establishment of the divisional molecular binding interaction(s). By controlling these variables it is possible to create domains in the sub-micron to many micron diameter size range. As may be appreciated, the locally homogeneous pools of material are randomly distributed on the surface. This process can be scaled down to produce locally homogeneous pools of defined molecular species on the nanometer size scale and containing from 1 to several thousand biomolecules (e.g., 60 Kd protein). The above steps create random distributions of locally homogeneous deposition domains containing deposition materials that create a site for molecular interaction detection by the AFM, and a target for the desorption of a uniform deposition material which is then analyzed for example by MS.

Initial Scan

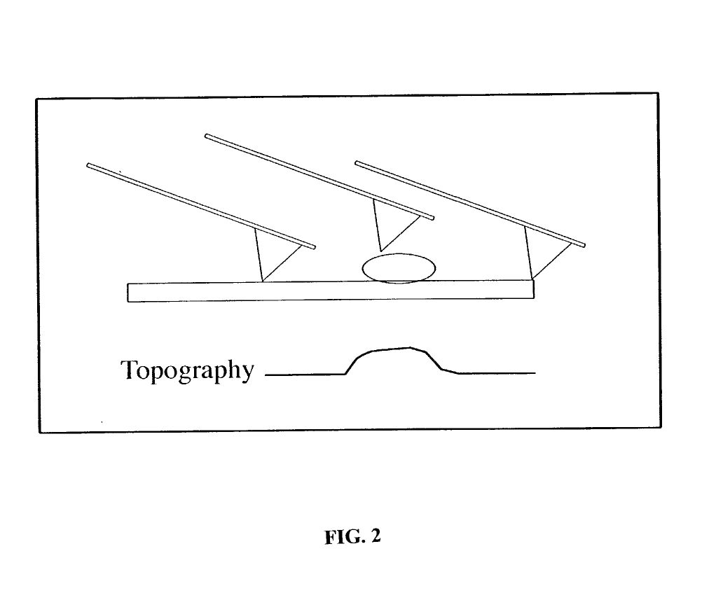

In the next step of the present invention method, the AFM may be utilized to scan (14) across the one or more deposition domains of biomaterials deposited on the substrate. The AFM may scan a 1 cm square chip or larger in sections of approximately 100μ² per section. Within each 100μ² scan exist one or more domains representing one or more deposition materials. The AFM can scan on the entire 100μ² area in 1-5 minutes. Scanning the surface and recording the data gives the user a “read-out” of the topography of the randomly created surface. See FIG. 2. A clear picture of the surface characteristics of the deposition domains is then obtained, whether topographical or other, for comparison with the surface after the array is exposed to the target material containing the unknown.

Topography is scanned using a variety of AFM detection methods, such as surface topography, local friction, phase, amplitude, viscosity and other force-related parameters. Any one or combination of two or more scans can be used to create the initial image of the surface.

The initial and subsequent imaging processes can be carried out in real time since the AFM can scan in fluids and one can introduce materials to the scanning area at any time during the scanning process. In this embodiment, the AFM is operated in solution using a standard “fluid cell” (Digital Instruments/Veeco, Santa Barbara, Calif.). The sample is added through access ports in the cell and the changes in surface topography noted. The locations of these changes are indexed with respect to physical markers such as alphanumerical etchings or marking introduced by the AFM probe that will allow precise relocation in the MS.

Exposure of the Target Sample

In the next step, the deposition domains are exposed to the target sample (16) containing the target material. The exposure of the target sample may be done by any manner known to those skilled in the art, such as with an aerosol spray, exposure to a gas, or by immersing the chip in the target solution. As may be appreciated, the target material is not limited to liquids, but may also be gases, granular solids, or other materials which can be exposed to a surface and result in a molecular interaction event.

Once the array and deposition materials are exposed to the target sample and the target material, the array is rinsed. Rinsing the surface will clear off any extraneous materials that were not actually the subject of a molecular interaction event. In some studies, the rinsing step may be excluded if the non-specific “clinging” type interactions are of interest, such as with weak biological interactions. In still another alternative embodiment, the force used during the below scanning process can be altered to remove materials bound with various (lower) affinities in a controlled fashion. This process, “force panning” is described elsewhere. See U.S. pending patent application Ser. No. 09/974,757, which is incorporated herein for all it teaches and discloses. The rinsing step can be accomplished by rinsing the array with water or some other solution such as a buffered saline solution. It is important to not desorb the target materials of interest, so the material used to rinse off the array should be carefully selected so as not to interfere with molecular interaction events that are of interest.

Second Scan

In the next step, the AFM is again utilized to scan the deposition domains on the surface of the array. During this second scan, molecular interaction events may be located by the change in topography, binding affinity, or other characteristic, as determined by the first scan. The places where the topography, has changed since the first scan implies that a molecular interaction event, such as binding by the target material, has taken place. The spatial location of each of these molecular interaction events may then be recorded and the information sent to the MS instrument.

The topography changes detected by the second AFM scan must be recognized by the mass spectrometer. Two general approaches to accomplishing this goal can be taken.

In the first general approach, the AFM can be integrated directly with the MS device. In this case, the AFM is operated in the ultrahigh vacuum chamber in which MS operates such has been described Gillen, G., J. Bennett, et al. (1994). Molecular imaging secondary ion mass spectrometry for the characterization of patterned self-assembled monolayers on silver and gold. Analytical Chemistry. 66: 2170-2174. Tang, K., D. Fu, et al. (1995). Matrix-assisted laser desorption/ionization mass spectrometry of immobilized duplex DNA probes. Nucleic Acids Research. 23: 3126-3131. Tarlov, M. J. and J. G. Newman (1992). Static secondary ion mass spectrometry of self-assembled alkanethiol monolayers on gold. Langmuir. 8: 1398-1405.

In this embodiment, the AFM scanner is contained within the MS chamber. Similar configurations have been described for AFMs integrated with electron microscopes Walters, D. A., D. Hampton, et al. (1994). Atomic force microscope integrated with a scanning electron microscope for tip fabrication. Applied Physics Letters. 65: 787-789.

Various configurations and methods can be utilized herein, such as scanning the deposition domains on the surface in a vacuum, exposing the array to the target sample in some biologically relevant medium, and then scanning the surface again in a vacuum, Alternatively, all of the scanning and exposure of the deposition domains may be carried out in the biologically relevant medium prior to evacuation of the MS chamber followed by exposure of the array to a vacuum when the surface is to be desorbed. As may be appreciated, a number of possible set-ups may be utilized without changing the basic invention.

In the above case there are two noteworthy comments. First, since this process is carried out in vacuum, it may be necessary to do the preliminary and secondary scans with the AFM under ambient condition to allow for the presence of a significant aqueous environment to facilitate the molecular binding events. Secondly, it may be desirable in some cases to include in the process the addition of a matrix to assist the desorption process, as previously described Girault, S., G. Chassaing, et al. (1996). Coupling of MALDI-TOF mass analysis to the separation of biotinylated peptides by magnetic streptavidin beads. Analytical Chemistry. 68: 2122-2126. Such a matrix may be underlayed or overlaid on the surface using methods known to those skilled in the art.

An alternative embodiment to desorbing using the MS laser is to utilize a conductive AFM probe (fabricated from conductive silicon or coated with a conductive metal) or an STM probe to introduce a high energy field at the location of the target material. Spence, J., U. Weierstall, et al. (1996). Atomic species identification in scanning tunneling microscopy by time of flight spectroscopy. J. Vac. Sci. Tech. B14(3), 1587-1590. Weierstall, U., and J. Spence. (1998). Atom species identification in STM using an Imaging Atom-Probe technique. Surface Science. 398:267-279. Ding, Y., M. Ruggero, et al. (2000). Development of UHV-STM/TOF Hybrid Mass Analyzer System for Nano-Characterization of Metal Silicide Surfaces. The 198^{th} Meeting of The Electrochemical Society, Phoenix, Ariz.

This energy field, created with a voltage bias or thermal heating, can result in ionization and desorption of materials from the domain and subsequent sensing by the MS apparatus. To facilitate the desorption process this voltage pulse may be supplemented with a laser pulse (Ding, Y., T. Oka, et al. (2001). Near-field stimulated TOF nanometric surface mass spectroscopy: characterization of Nano-localized surfaces. 2001 Joint International Meeting—the 200th Meeting of The Electrochemical Society, Inc. and the 52nd Annual Meeting of the International Society of Electrochemistry, San Francisco, Calif.

A further method is to use an NSOM probe to create a local laser pulse and desorb materials into a local “sniffer” pipette which then directs the materials to the MS detector Stöckle, R., P. Setz, et al. (2001). Nanoscale Atmospheric Pressure Laser Ablation-Mass Spectrometry. Alternatively, direct physical contact, or “tapping” of the surface with the scanning probe may result in sufficient desorption to allow MS detection of the released materials. Any method for imparting energy to the surface causing desorption of materials from the surface into the MS detector may be employed as necessary to accomplish this goal.

In the second general approach, the entire AFM process may be carried out in a more convenient environment, such as on a desktop using a conventional AFM (e.g., Dimension 3100, Digital Instruments/Veeco, Santa Barbara, Calif.). In this embodiment, the first and second AFM scans are taken on an indexed surface (20). The indexing marks or features are then used to re-locate the precise positions of the molecular interactions of interest and acquire MS data from these locations. To accomplish this, the surface must be indexed in some fashion so that the spot to be analyzed by MS can be relocated once the sample is introduced into the MS device. In one approach, the random distribution of materials can be carried out on an indexed surface containing markers at sufficient regularity to allow one to locate with certainty the same spot after introduction into the MS device. Such indexed surfaces include surfaces containing gold or another metal sputtered through a mask of the appropriate dimensions. An example of such a mask is a standard alpha numeric indexed electron microscopy grid (Electron Microscopy Sciences, Fort Washington, Pa.). Alternatively, glass or silicon surfaces can be etched by methods known to those in the art to create index marks of sufficient resolution. A second method for indexing is to use the AFM (or other scan probe) as an etching tool and score the area around the site of interest. In this way, a defining scoring pattern is created to allow precise localization of the spot of interest. Jin, X., and W. Unertl. (1992). Submicrometer modification of polymer surfaces with a surface force microscope. Applied Physics Letters. 61:657-659. Magno, R., and B. Bennett. (1997). Nanostructure patterns written in III-V semiconductors by an atomic force microscope. Applied Physics Letters. 70:1855-1857. An optical system is then incorporated into the MS device to allow precise localization of the laser for desorption and subsequent MS detection and analysis.

Mass Spectrometry

Once domains of interest are identified they are interrogated by MS (22) (or AFM). Because of the size and localization of the domains, the spectrometer can desorb the material from the surface with a reasonable likelihood that only one type of modified deposition material is being desorbed and analyzed. The desorbed material may be analyzed utilizing the MS with a mass detector, gas chromatograph, or other ways known to those of skill in the art. The analysis from the MS may be able to identify the target unknown. For example, mass spectra can be considered to be signatures for particular molecular species.

In the case of known proteins, for example, it is possible to predict the mass spectra for each protein and look for those mass identifiers in the mass spectrum from materials desorbed from a particular domain. In this case, which proteins form the detected complex are not necessarily “known”, but instead a defined set of proteins is tested and so the protein is “known” in the sense that it must be part of that set. The spectrum by the test can therefore be compared with the spectra of the “known” set. In the case where the molecular species being examined are not known, the mass spectra can be used to determine features of the proteins or other biomaterials being analyzed if not their precise identity. There is some likelihood that the mass spectrum will contain data from both the deposition material and the target material bound thereon. However, since the class of deposition materials is generally known, though the locations are not, it is possible to separate out those contributions from the deposition material those contributions to the mass spectra that arise from the target material.

As may be appreciated, the present embodiment may utilize a programmable computer to carry out the present invention steps. The present invention apparatus may also electronically record the position of each molecular interaction event. In yet another embodiment after detection of molecular interactions, the energy from the near field probe (e.g., a scanning tunneling microscope probe using a voltage bias) is used to selectively desorb materials. The materials are then detected using mass spectrometer methods.

### EXAMPLE 1

Antibody antigen interactions. In this example a variety of antibodies are randomly distributed in deposition domains on a surface by the methods described herein. The surface thus constructed is interrogated using a target sample solution that may contain some or all of the antigens that react with the deposited antibodies. Upon incubation with these materials in a standard biological buffer (phosphate buffered saline, pH 7.2) at a concentration of 0.1 mg/ml protein for 30 minutes at room temperature the antibodies on the surface react with the antigens present in the mixture.

In this example, the antibodies are monoclonal antibodies directed against the protein cytokines interferon-gamma (IFN-g) and interleukin 6 (IL6). The antibodies are deposited as described herein. The surface is incubated for 2 hours with a solution containing IFN-g (10 ng/ml) in 10 mM Tris-HCl (pH 7.2), 10 mM NaCl. The reacted surface is washed 4 times with 1 ml of the incubation buffer minus the IFN-g. An AFM scan of the surface after this interaction is compared to one taken before this interaction and those regions where binding events have occurred are noted. Indexing marks are created by the AFM probe or are preexisting on the surface. This is accomplished by increasing the force and creating lines and squares by scanning small domains at the periphery of the targets that are selected based on topographic changes. These marks are visible optically and are used to re-locate the sites of molecular interactions between the anti-IFN-g and the IFN-g. MS spectra are taken from these location using a minimal laser spot size (1-5μ) and the IFN-g MS signature spectrum sought. Control sample spectra are taken at other location where molecular binding and topographic changes were not observed to establish a background level of signal. In this example, the spectrum for IFN-g is found in locations as expected and not in locations where no topographic changes were observed by AFM. Additionally, no signal for IL6 is found at any location since this cytokine was not in the initial sample mixture, although anti-IL6 antibodies were present on the surface.

### EXAMPLE 2

Protein-protein interactions for proteomics. In this example the goal is to determine which proteins interact with which other proteins in a complex proteome. The yeast proteome serves as an example. The ˜6000 gene products from the yeast proteome are distributed randomly as described herein on a surface. A non-ionic detergent such as Tween 20 may be included in the deposition process if desired to minimize chances of undesirable protein-protein interactions occurring during surface construction. The surface thus constructed is imaged by AFM, then interrogated with from one to hundreds of the same proteins one at a time or in a complex mixture. Binding events occur and are identified by AFM or some other scanning probe method. The binding events are carried out in a standard binding buffer such as Tris-HCl, pH 7.2, 100 mM NaCl, 1 mM EDTA, 1 mM MgCl₂ or any other binding buffer. It is noteworthy that varying the binding conditions is a valuable parameter in examining a variety of classes of binding interactions. The identified binding domains are characterized by MS and the resulting spectrum used to identify those proteins that interact under the given set of binding conditions.

The AFM has been used as the general method for measuring binding interactions but as is evident to those skilled in the art, a variety of other methods may be substituted for the AFM to accomplish the same goals. For example a fluorescently labeled library of antigens or proteins may be used in the examples above to identify binding domains, which are then interrogated by MS. Alternatively, quantum dots, resonance light scattering particles, enzyme reactions, radioactivity, Raman or infra red spectroscopy, or other methods may be used in place of AFM for the initial localization of binding events. In some cases these methods can also facilitate or corroborate the MS spectra-based identification of the molecular species participating in the binding reactions.

It is contemplated that various changes can be made to the present invention without deviating from the scope of the present invention as described herein and in the accompanying drawings. Therefore, it is desired that the described embodiments be considered in all respects as illustrative, not restrictive.

Although the present invention has been described with reference to preferred embodiments, persons skilled in the art will recognize that changes may be made in form and detail without departing from the spirit and scope of the invention.

I claim:

1. A method for detecting a target material comprising:
depositing a deposition material on a surface;
exposing the deposition material to a target sample wherein the target sample contains one or more target materials that are to be characterized, the target material interacting with the deposition material on the surface to form a reaction product on the surface;
scanning the surface with a scanning probe microscope to locate the position of the reaction product on the surface;
desorbing the reaction product from the surface; and
detecting the desorbed reaction product.

2. The method of claim 1 further comprising analyzing an output from a mass spectrometer to characterize the reaction product desorbed from the surface.

3. The method of claim 1 wherein the reaction product is desorbed from the surface using a laser.

4. The method of claim 1 wherein the reaction product is desorbed from the surface using a probe attached to the scanning tunneling microscope.

5. The method of claim 1 wherein the scanning probe microscope is an atomic force microscope.

6. The method of claim 1 in which the scanning probe microscope is a near field optical microscope.

7. The method of claim 1 wherein the molecular interaction event includes one or more, of physisorption are hydrogen binding.

8. The method of claim 1 wherein the deposition the deposition material on the surface forms a deposition domain.

9. The method of claim 1 wherein the deposition material is an antibody.

10. The method of claim 1 wherein the deposition material is a protein.

11. The method of claim 1 wherein the target material is chosen from the group consisting of a protein, an antibody, a pathogen, and a virus.

12. The method of claim 1 wherein the surface includes a substrate.

13. The method of claim 12 wherein the substrate is chosen from one of the group consisting of glass, silicon, mica, and quartz.

14. The method of claim 1 wherein depositing the deposition material on the surface further comprises coating the surface with gold.

15. The method of claim 14 wherein depositing the deposition material on the surface further comprises attaching a C11-C18 alkane linker to the gold layer.

16. The method of claim 15 wherein the alkane linker includes a functional portion selected from a group consisting of a COOH, a NH2, and a succinimide.

17. The method of claim 15 wherein the alkane linker interacts with a primary amine of a protein.

18. The method of claim 1 wherein depositing the deposition material further comprises spraying the deposition material on the surface.

19. The method of claim 18 wherein the deposition material is a liquid.

20. The method of claim 18 wherein the deposition material is dissolved or suspended in a liquid.

21. The method of claim 18 wherein the material is sprayed as an aerosol spray.

22. The method of claim 1 wherein depositing the material on the surface further comprises:
crystallizing the deposition material;
depositing the crystallized deposition material on the surface;
hydrating the crystallized deposition material deposited on the surface.

23. The method of claim 22 wherein depositing the deposition material includes forming a deposition domain.

24. The method of claim 1 wherein depositing the deposition material on the surface further comprises:
placing the surface at the bottom of a coating of liquid nitrogen;
freezing the deposition material in the liquid nitrogen and letting it fall to the surface;
removing the surface from the liquid nitrogen;
hydrating the deposition material.

25. The method of claim 24 wherein depositing the deposition material further comprises forming a deposition domain.

26. An apparatus for the detection and characterization of molecular interaction events comprising:
a scanning tunneling microscope; and
a mass spectrometer.

27. The apparatus of claim 26 wherein the mass spectrometer is operably integrated with the scanning tunneling microscope wherein the scanning tunneling microscope detects an interaction between a deposition material deposited on a surface and a target material and wherein at least the target material is desorbed from the surface and analyzed by the mass spectrometer.

28. A method for determining protein interactions
distributing randomly on a surface a first set of proteins;
imaging the randomly distributed proteins on the surface;
exposing the first set of proteins on the surface to a second set of proteins;
imaging the protein-protein interactions;
noting the location of the protein-protein interactions;
characterizing the protein-protein interactions using mass spectrometry.

29. The method of claim 28 wherein imaging is accomplished using a scanning probe microscope.

30. The method of claim 29 wherein the scanning probe microscope is an atomic force microscope.
